(19) United States
(12) Patent Application Publication
(10) Pub. No.: US 2017/0209164 A1
(43) Pub. Date: Jul. 27, 2017

(54) MEDICAL TREATMENT TOOL

(71) Applicant: OLYMPUS CORPORATION, Tokyo (JP)

(72) Inventor: Noriaki YAMANAKA, Tokyo (JP)

(73) Assignee: OLYMPUS CORPORATION, Tokyo (JP)

(21) Appl. No.: 15/482,933

(22) Filed: Apr. 10, 2017

Related U.S. Application Data
(63) Continuation of application No. PCT/JP2014/078996, filed on Oct. 30, 2014.

(51) Int. Cl. A61B 17/29; B25J 15/00; A61B 1/018; A61B 34/00; A61B 34/37

(52) U.S. Cl. CPC A61B 17/29; A61B 34/71; A61B 34/37; A61B 34/77; A61B 1/018; B25J 15/0028; A61B 2017/2932; A61B 2090/034

(57) ABSTRACT

A medical treatment tool includes: a long flexible section; a movable member that is attached to a distal end of a flexible section so as to be relatively movable in a longitudinal-axis direction of the flexible section; a grasper that is attached to the movable member and that includes a pair of jaws that can be opened and closed, at least one of which is supported so as to be pivotable about a shaft intersecting the longitudinal axis; and a tension transmission disposed in the flexible section so as to be able to advance and retract in the longitudinal-axis direction, having one end joined to the jaws, and pivoting the jaws by transmitting the tension.

### CROSS-REFERENCE TO RELATED APPLICATIONS

This is a continuation of International Application PCT/JP2014/078996, with an international filing date of Oct. 30, 2014, which is hereby incorporated by reference herein in its entirety. This application claims the benefit of International Application PCT/ JP2014/078996.

### TECHNICAL FIELD

The present invention relates to medical treatment tools.

### BACKGROUND ART

A known medical treatment tool grasps tissue by closing, by means of pulling a wire, two jaws attached, in such a manner that they can be opened and closed, to the distal end of a sheath part attached to a slave arm (for example, see PTL

This medical treatment tool is a grasping forceps that enables the jaws to generate large grasping force, by means of a force boosting structure provided between the wire and the jaws, even when a small pulling force is applied to the wire.

### CITATION LIST

### Patent Literature

{PTL 1} Japanese Unexamined Patent Application, Publication No. 2012-187311

### SUMMARY OF INVENTION

### Technical Problem

An object of the present invention is to provide a medical treatment tool that enables a grasping forceps attached to the distal end of a flexible section to more reliably grasp tissue, even if the flexible section is bent.

### Solution to Problem

An aspect of the present invention is a medical treatment tool including: a long flexible section; a movable member attached to a distal end of the flexible section so as to be relatively movable in a longitudinal-axis direction of the flexible section; a grasper that is attached to the movable member and that includes a pair of jaws that can be opened and closed, at least one of the jaws being supported so as to be pivotable about a shaft intersecting the longitudinal axis; and a tension transmission that is disposed in the flexible section so as to be able to move forward and backward in the longitudinal-axis direction, that is joined to the jaws at one end, and that transmits tension to pivot the jaws.

In the above aspect, the movable member may include a first movable part that is attached to the flexible section so as to be relatively movable in the longitudinal-axis direction, and a second movable part that is attached to the first movable part so as to be relatively movable in the longitudinal-axis direction. In a state in which the grasper is maximally brought toward the flexible section, the first movable part, the second movable part, and the flexible section are stacked on top of one another in the longitudinal-axis direction.

In the above aspect, the medical treatment tool may further include an anchoring part that anchors the flexible section and the tension transmission when the flexible section is moved toward the proximal end relative to the movable member with the jaws of the grasper closed.

In the above aspect, the medical treatment tool may further include a restriction mechanism that restricts the amount of relative movement between the flexible section and the movable member.

In the above aspect, the tension transmission may include a pair of tension transmissions provided in accordance with the pivot directions of the jaws. The tension transmissions are driven such that, when the tension in one tension transmission is increased, the tension in the other tension transmission is reduced. The initial tensions in the tension transmissions may be set such that the difference in tension between the pair of tension transmissions is increased when the flexible section is moved toward the proximal end relative to the movable member.

### BRIEF DESCRIPTION OF DRAWINGS

FIG. 1 is a diagram showing the overall configuration of a medical system including a medical treatment tool according to an embodiment of the present invention.

FIG. 2 is a vertical sectional view of a distal end portion of the medical treatment tool according to the embodiment of the present invention.

FIG. 3 is a vertical sectional view of the distal end portion, showing a state in which a grasper of the medical treatment tool in FIG. 2 grasps tissue.

FIG. 4 is a graph showing the relationship between the tension in a wire and the position in the longitudinal direction of the wire of the medical treatment tool in FIG. 3.

FIG. is a vertical sectional view of a distal end portion, showing a state in which a flexible section of the medical treatment tool in FIG. 3 is moved toward the proximal end.

FIG. 6 is a graph showing the relationship between the tension in the wire of the medical treatment tool in FIG. 5 and the position in the longitudinal direction of the wire.

FIG. 7A is a vertical sectional view of a distal end portion, showing a state in which a grasper of a medical treatment tool according to a first modification of the embodiment of the present invention grasps tissue.

FIG. 7B is a vertical sectional view of a distal end portion, showing a state in which a flexible section of the medical treatment tool in FIG. 7A is moved toward the proximal end.

FIG. 8A is a vertical sectional view of a distal end portion of a medical treatment tool according to a second modification of the embodiment of the present invention.

FIG. 8B is a vertical sectional view of a distal end portion, showing a state in which the grasper of the medical treatment tool in FIG. 8A grasps tissue.

FIG. 8C is a vertical sectional view of a distal end portion, showing a state in which a flexible section of the medical treatment tool in FIG. 8B is moved toward the proximal end.

FIG. 9A is a vertical sectional view of a distal end portion of a medical treatment tool according to a third modification of the embodiment of the present invention.

FIG. 9B is a vertical sectional view of a distal end portion, showing a state in which the grasper of the medical treatment tool in FIG. 9A grasps tissue.

FIG. 9C is a vertical sectional view of a distal end portion, showing a state in which a flexible section of the medical treatment tool in FIG. 9B is moved toward the proximal end.

FIG. 10A is a vertical sectional view of a distal end portion of a medical treatment tool according to a fourth modification of the embodiment of the present invention.

FIG. 10B is a vertical sectional view of a distal end portion, showing a state in which the grasper of the medical treatment tool in FIG. 10A grasps tissue.

FIG. 10C is a vertical sectional view of a distal end portion, showing a state in which a flexible section of the medical treatment tool in FIG. 10B is moved toward the proximal end.

FIG. 11 is a graph showing the relationship between the tensions in two wires of the medical treatment tool in the state in FIG. 10B and the positions in the longitudinal direction of the wires.

FIG. 12 is a graph showing the relationship between the tensions in the two wires of the medical treatment tool in the state in FIG. 10C and the positions in the longitudinal direction of the wires.

### DESCRIPTION OF EMBODIMENTS

A medical treatment tool 1 according to an embodiment of the present invention will be described below with reference to the drawings.

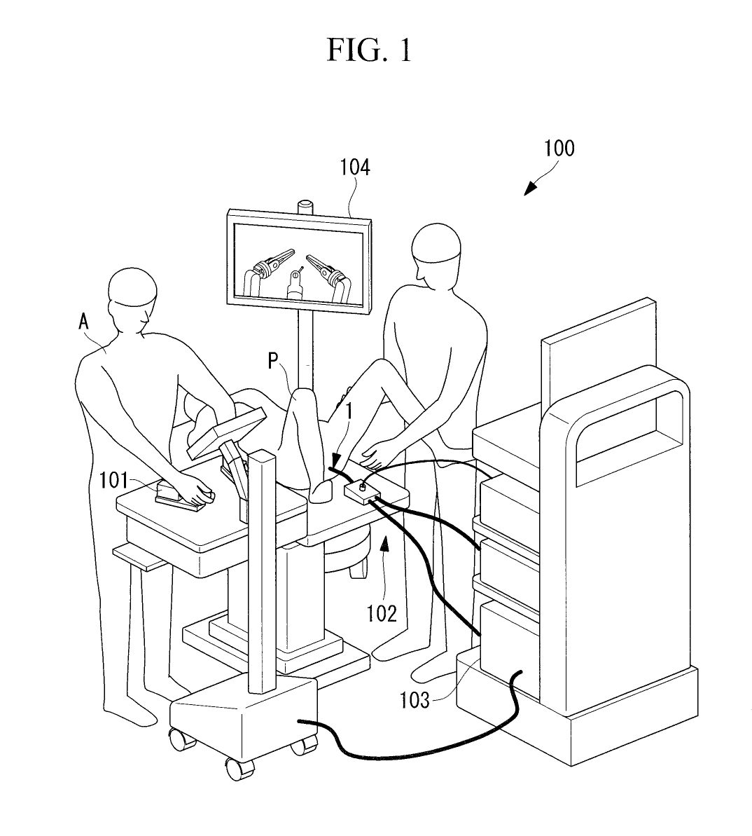

The medical treatment tool 1 according to this embodiment is applied to, for example, a medical system 100 shown in FIG. 1.

The medical system 100 includes: a master device 101 operated by a doctor A; a slave device 102 driven according to the input via the master device 101; a controller 103 that controls the slave device 102 on the basis of the input to the master device 101; and a monitor 104. The slave device 102 includes an insertion part to be inserted into a body cavity of a patient P. The medical treatment tool 1 according to this embodiment is provided at the distal end of the insertion part.

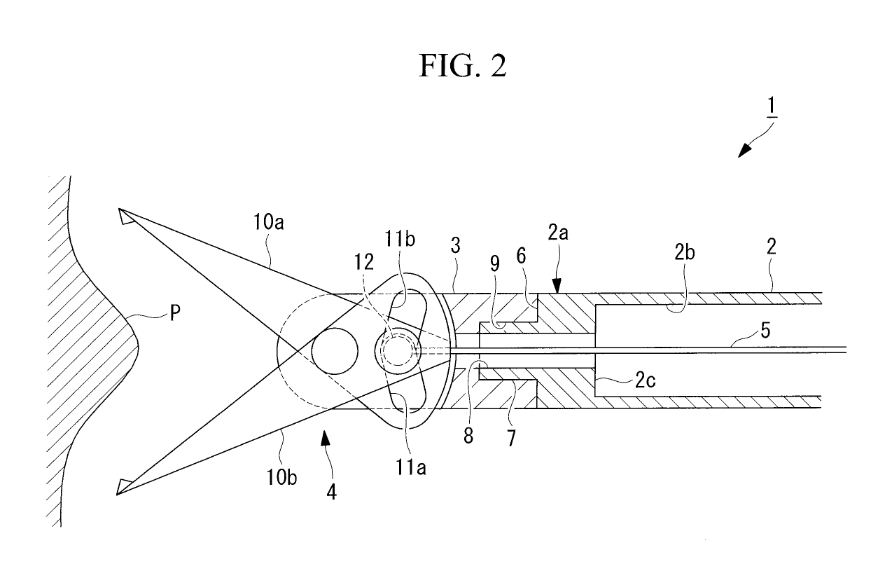

As shown in FIG. 2, the medical treatment tool 1 according to this embodiment is a grasping forceps and includes: a long flexible section 2; a movable member 3 attached to a distal end 2a of the flexible section 2; a grasper 4 attached to the movable member 3; and a wire (tension transmission) 5 that passes through the flexible section 2 and the movable member 3 in the longitudinal-axis direction. One end of the wire 5 is connected to the grasper 4, and the other end (not shown) of the wire 5 is pulled at the proximal end of the flexible section 2.

The flexible section 2 is configured as a hollow tube and includes, near the distal end 2a, a bending section (not shown) that bends the distal end portion about an axis intersecting the longitudinal axis. The wire 5 is disposed inside the flexible section 2 so as to pass through the flexible section 2 along the longitudinal axis. An end face 6 of the flexible section 2 is provided with a columnar projection 7 extending in the longitudinal-axis direction. A through-hole 8, through which the wire 5 passes, is provided at the center of the projection 7.

The movable member 3 has a fitting hole 9, into which the projection 7 on the end face 6 of the flexible section 2 is fitted. By fitting the projection 7 into the fitting hole 9 so as to be movable in the longitudinal direction, the movable member 3 is attached to the flexible section 2 so as to be movable in the longitudinal direction.

The grasper 4 includes a pair of jaws 10a and 10b attached to the movable member 3 so as to be pivotable about a pivot shaft perpendicular to the longitudinal axis of the flexible section 2.

In the example shown in FIG. 2, the pair of jaws 10a and 10b have, at positions closer to the proximal end than the pivot shaft is, elongated holes 11a and 11b, respectively, that are with respect to the longitudinal directions of the jaws 10a and 10b. The elongated holes 11a and 11b provided in the pair of jaws 10a and 10b are inclined in the opposite directions, and a pin 12, to which one end of the wire 5 is fixed, is disposed so as to pass through the two elongated holes 11a and 11b.

Specifically, when the wire 5 is pulled toward the proximal end, the pin 12 fixed to one end of the wire 5 is moved toward the proximal end. As a result, the positions of the elongated holes 11a and 11b are moved so as to allow the movement of the pin 12. Specifically, the jaws 10a and 10b provided with the elongated holes 11a and 11b are pivoted about the pivot shaft in the opposite directions, whereby the pair of jaws 10a and 10b are closed to grasp tissue P or the like therebetween. Note that a spring (not shown) is disposed between the pair of jaws 10a and 10b, and, in a state in which no tension is applied to the wire 5, the resilient force of the spring acts in the directions in which the pair of jaws 10a and 10b are opened.

The operation of the thus-configured medical treatment tool 1 according to this embodiment will be described below.

When tissue P inside the body of a patient is to be treated with the medical treatment tool 1 according to this embodiment, the movable member 3 is maximally moved toward the flexible section 2, and an initial tension exceeding the resilient force of the spring is applied to the wire 5 to close the pair of jaws 10a and 10b. Then, the medical treatment tool 1 is inserted into the body from the grasper 4 side through a guide sheath or a forceps channel of an endoscope (not shown). Then, the grasper 4 is projected from the guide sheath or a distal-end opening of the forceps channel and is positioned to face the tissue P inside the body.

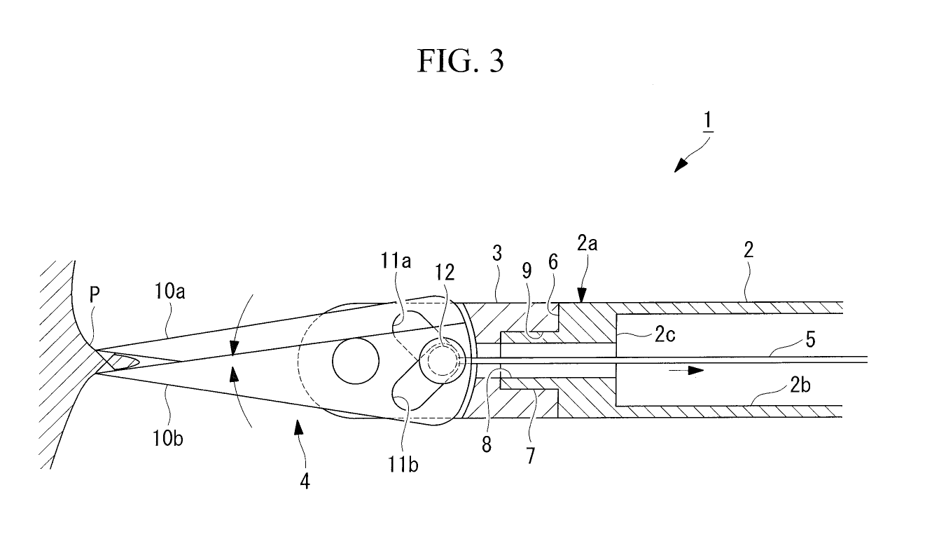

By reducing the tension applied to the wire 5 with the grasper 4 facing the tissue P, as shown in FIG. 2, the pair of jaws 10a and 10b are opened, and the tissue P is positioned between the jaws 10a and 10b. Then, by pulling the wire 5 at the proximal end of the flexible section 2 to generate tension in the wire 5, as shown in FIG. 3, the pair of jaws 10a and 10b are closed to grasp the tissue P therebetween.

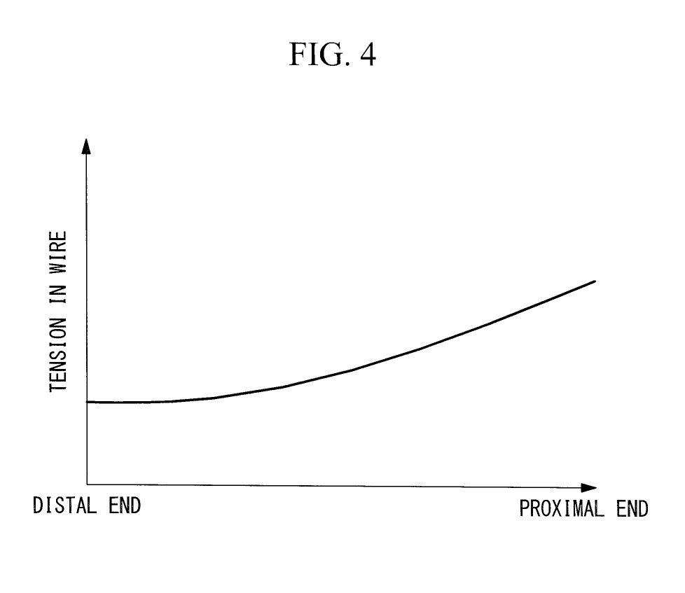

When the flexible section 2 is bent so as to conform to the shapes of the body cavities and the organs before the grasper 4 is positioned at the site to be treated, the tension in the wire 5 generated by applying a pulling force at the proximal end of the flexible section 2 is attenuated due to the friction between the flexible section 2 and the wire 5 near the grasper 4 provided at the distal end 2a of the flexible section 2. Thus, as shown in, for example, FIG. 4, the magnitude of the tension in the wire 5 varies among portions in the length direction of the wire 5.

Accordingly, as long as the tension in the wire 5 at the distal end 2a of the flexible section 2 has the minimum necessary magnitude for pivoting the jaws 10a and 10b against the resilient force of the spring, the jaws 10a and 10b are closed to grasp the tissue P. However, if the tension is reduced to some degree by some cause, the grasped state could be terminated.

To counter this situation, in this embodiment, from the state shown in FIG. 4, by applying, at the proximal end of the flexible section 2, a force for pulling the flexible section 2 toward the proximal end, the flexible section 2 is moved toward the proximal end, relative to the movable member 3, as shown in FIG. 5.

Specifically, the movable member 3 is attached to the flexible section 2 so as to be movable in the longitudinal- axis direction of the flexible section 2 through the configuration in which the fitting hole 9 receives the projection 7 on the distal end 2a of the flexible section 2. Hence, by moving the flexible section 2 toward the proximal end with the grasper 4 grasping the tissue P, the flexible section 2 alone can be moved toward the proximal end, almost without changing the position of the grasper 4.

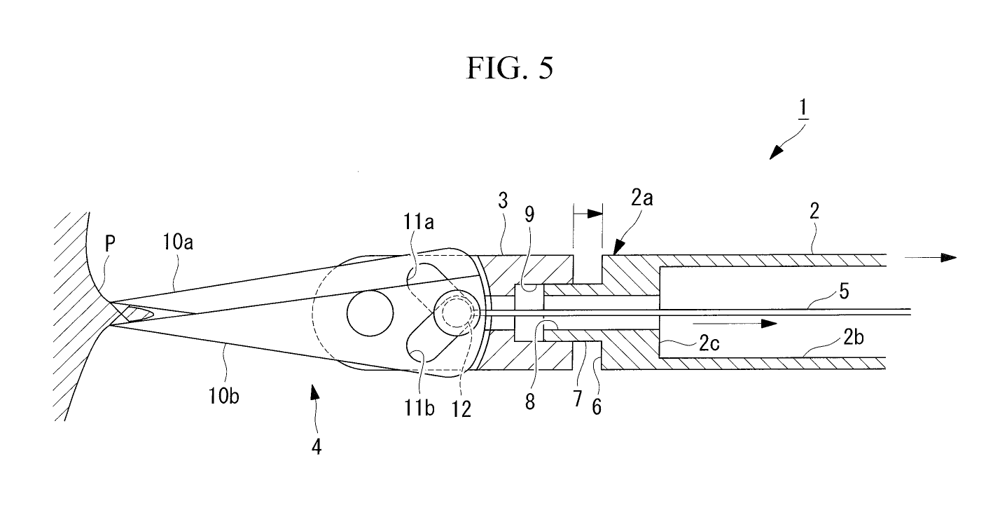

As a result, as shown by a solid line in FIG. 6, the tension in a portion of the wire 5 located near the distal end 2a of the flexible section 2 is increased.

Specifically, the portion of the wire 5 located near the distal end 2a of the flexible section 2 is fixed due to the friction with respect to an inner wall 2b of the flexible section 2 by being brought into close contact with the inner wall 2b of the flexible section 2 at some position at the proximal end, and thus, the tension is less likely to be transmitted to a portion closer to the distal end 2a than that position is.

To counter this situation, by pulling the flexible section 2 toward the proximal end, the distance between the grasper 4 and the position where the wire 5 is in close contact with the flexible section 2 is increased, and thus, the tension applied to the wire 5 can be increased.

This leads to an advantage in that it is possible to increase the force for closing the jaws 10a and 10b and, thus, to prevent the grasped state from being terminated when the tension is reduced to some degree by some cause.

In this embodiment, although the movable member 3, which supports the grasper 4, is provided with the fitting hole 9, and the projection 7, which is movably fitted into the fitting hole 9, is provided in the flexible section 2, the relationship between the fitting hole 9 and the projection 7 may be reversed.

In this embodiment, although an exemplary case where the pair of jaws 10a and 10b, which constitute the grasper 4, are both pivotably attached to the movable member 3 has been described, instead, it may be configured such that one jaw 10b is fixed, and only the other jaw 10a is pivotably attached.

In this embodiment, although an example configuration in which a spring is disposed between the pair of jaws 10a and 10b has been described, instead, a configuration in which no spring is disposed between the pair of jaws 10a and 10b may be employed.

In that case, it may be configured such that the jaws 10a and 10b are opened by pushing the wire 5 toward the distal end 2a, along the longitudinal axis. It is desirable that the friction between the movable member 3 and the flexible section 2 be greater than the pushing force applied to the wire 5 to open the jaws 10a and 10b. By doing so, the movable member 3 is prevented from moving toward the distal end 2a when the jaws 10a and 10b are opened.

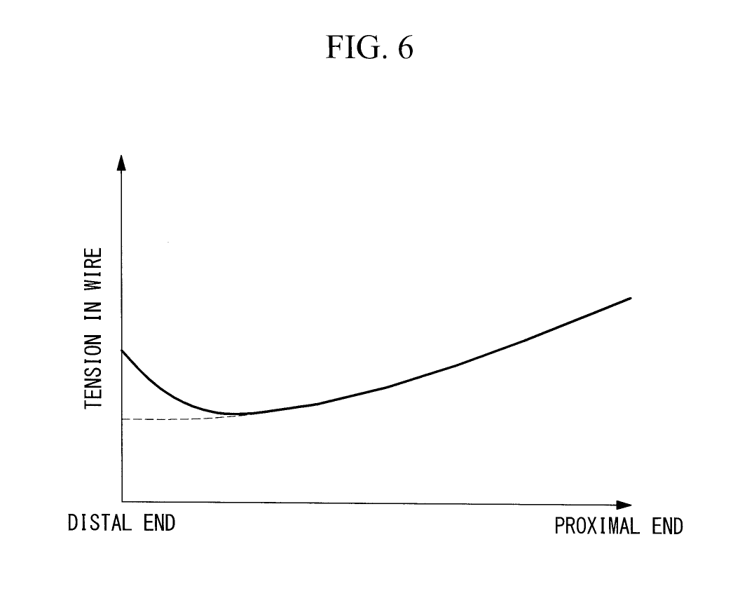

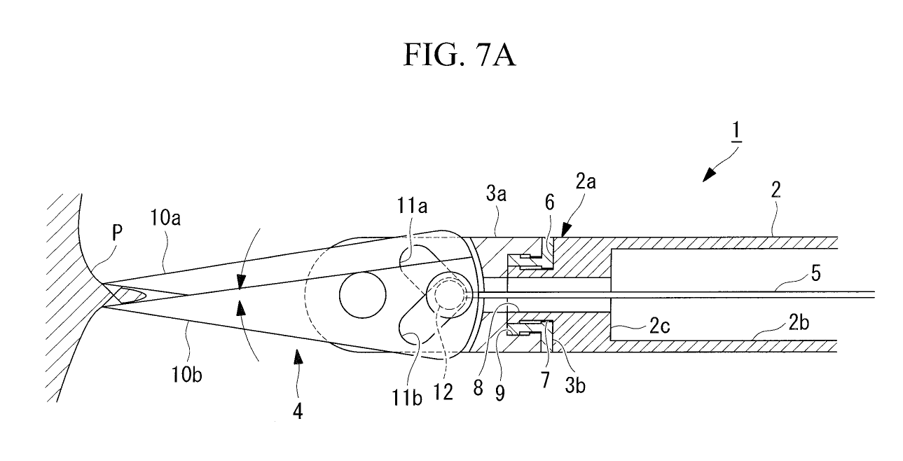

Although the movable member 3 and the flexible section 2 are joined together so as to allow relative movement by fitting the single projection 7 into the single fitting hole 9, instead, as shown in FIG. 7A and FIG. 7B, it is possible to employ a structure, for example, a telescopic structure, in which the movable member 3 is divided into a plurality of parts, and, in a state in which a movable member 3a and the flexible section 2 are relatively moved to positions where the grasper 4 is closest to the flexible section 2, the plurality of movable members (first and second movable parts) 3a and 3b are disposed at positions overlapping the flexible section 2 in the longitudinal direction.

With this configuration, it is possible to reduce the lengths of the movable members 3a and 3b in the longitudinal-axis direction in a state in which the grasper 4 is closest to the flexible section 2, while ensuring the same amount of relative movement as that in the case where the single projection 7 is fitted into the single fitting hole 9. This leads to an advantage in that it is possible to reduce the length of a non-bendable rigid section disposed in front of the bending section, facilitating movement of the medical treatment tool 1 along a winding path. Although an exemplary case where the number of movable members is two has been described, instead, it is possible to employ a configuration in which there are three or more movable members.

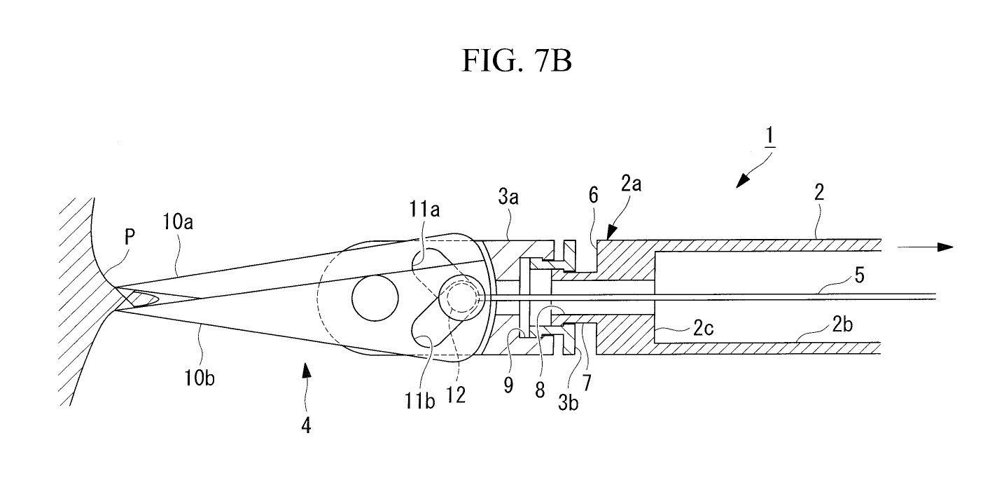

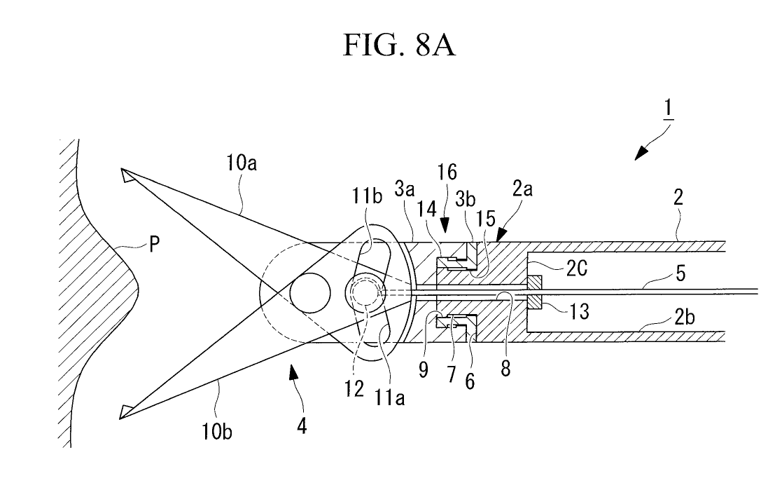

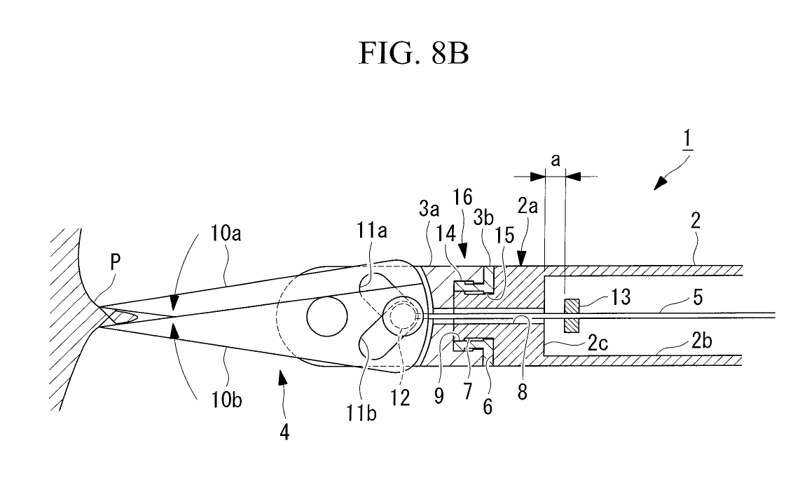

An anchoring part 13 projecting in the radial direction so as to be larger than the diameter of the through-hole 8 in the distal end 2a of the flexible section 2, through which the wire 5 passes, may be fixed to a portion of the wire 5 closer to the proximal end than the through-hole 8 is. As shown in, for example, FIG. 8A, the anchoring part 13 is disposed at a position where it abuts on an abutting face 2c on the proximal end side of the flexible section 2, in a state in which the grasper 4 is opened.

In this case, as shown in FIG. 8B, when the grasper 4 is closed by pulling the wire 5 toward the proximal end, a gap a is formed between the anchoring part 13 and the abutting face 2c. The maximum amount of relative movement between the movable member 3 at the extreme distal end and the flexible section 2 in this case is set to be greater than the gap a. In the example shown in FIG. 8C, the medical treatment tool 1 is configured such that, in a state in which the flexible section 2 is pulled toward the proximal end, relative to the movable member 3, and the gap a is eliminated, the flexible section 2 can be further relatively moved by a distance b.

With this configuration, when the flexible section 2 is pulled toward the proximal end, relative to the movable member 3, the anchoring part 13 comes into contact with the abutting face 2c of the flexible section 2 during pulling, and the pulling force acting on the flexible section 2 starts to directly act on the wire 5 between the anchoring part 13 and the jaws l0a and 10b. As a result, it is possible to more reliably increase the tension in the wire 5 near the jaws l0a and 10b and, thus, to maintain the jaws 10a and 10b in a closed state.

In the examples shown in FIG. 8A, FIG. 8B, and FIG. 8C, the movable member 3 is divided into a plurality of parts, and outer flange parts 14 and inner flange parts 15, which engage with one another, are provided between the movable members 3a and 3b, so that the movable members 3a and 3b are moved in an associated manner when the flexible section 2 is pulled. By doing so, when the flexible section 2 is pulled toward the proximal end, the plurality of movable members 3a and 3b are moved in an associated manner, and thus, relative movement between the flexible section 2 and the movable member 3a becomes possible. At the same time, when all the outer flange parts 14 and the inner flange parts 15 engage with one another, further relative movement between the flexible section 2 and the movable member 3a is restricted. Hence, the outer flange parts 14 and the inner flange parts 15 constitute a restriction mechanism 16.

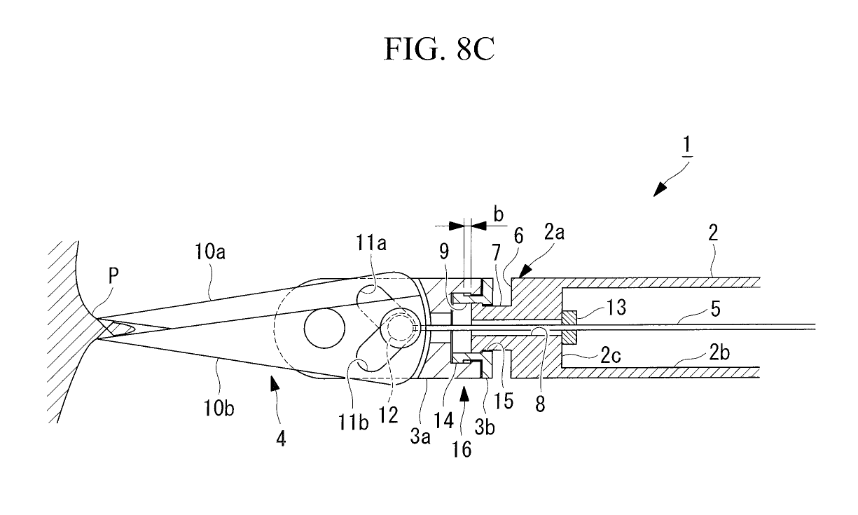

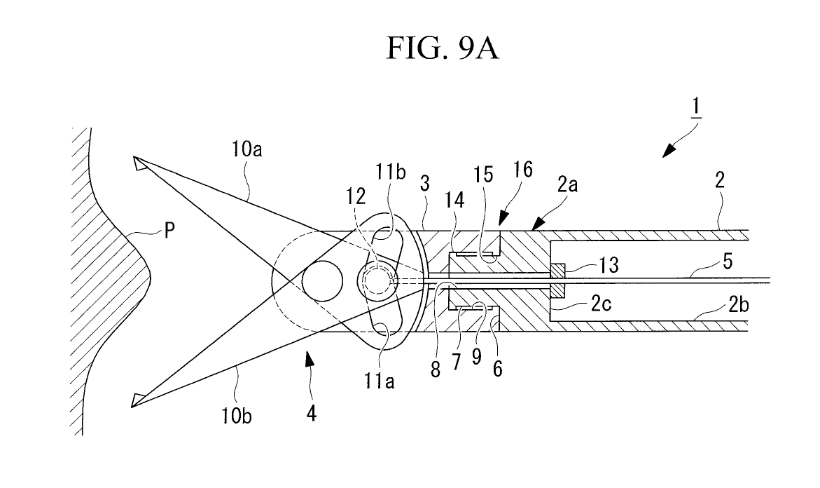

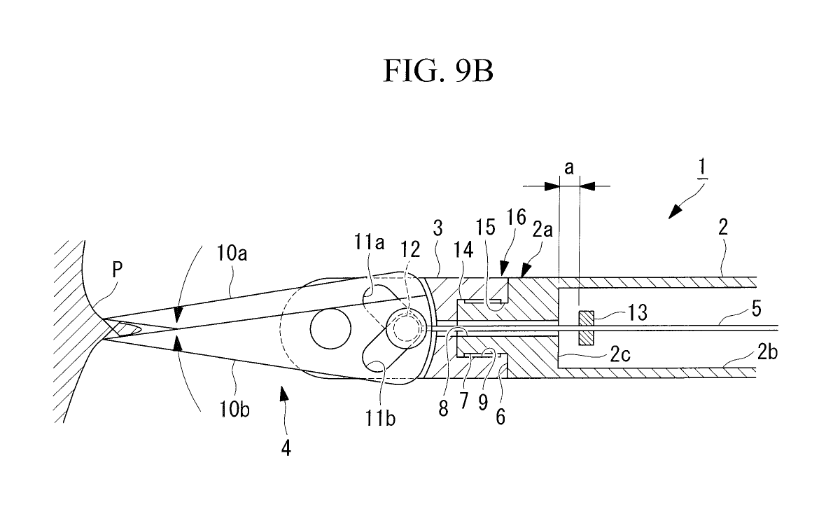

The provision of the restriction mechanism 16 makes it possible to prevent excessive tension from being generated in the wire 5 and, thus, to maintain the soundness of the wire 5. As shown in FIG. 9A, FIG. 9B, and FIG. 9C, the restriction mechanism 16 may also be provided when the movable member 3 is a single component.

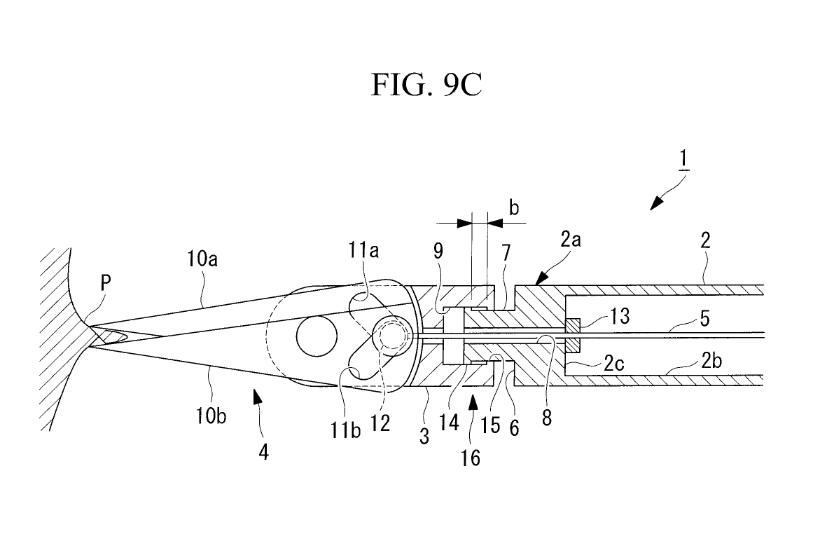

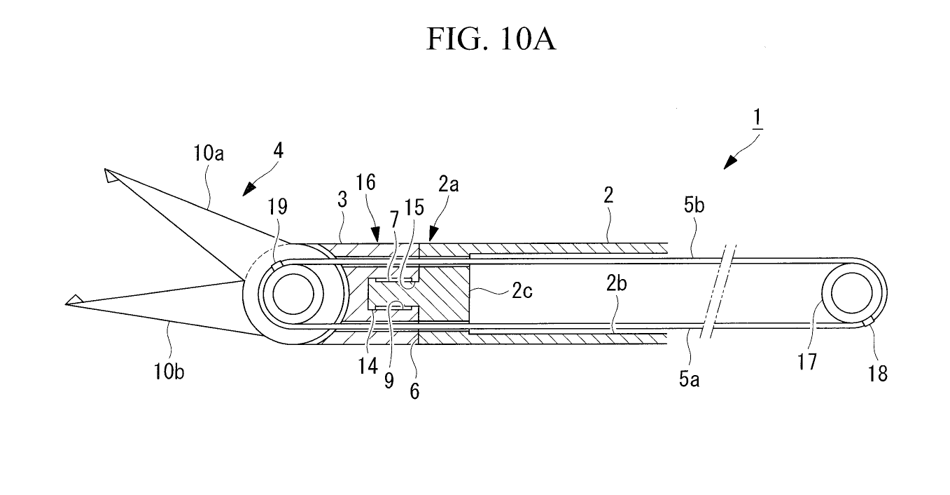

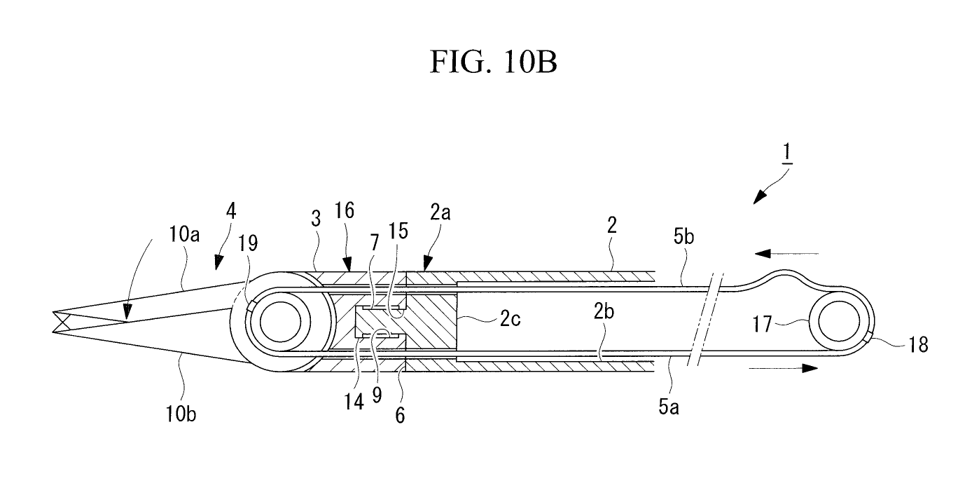

In this embodiment, although an exemplary configuration in which the grasper 4 is opened and closed by the single wire 5 has been described, instead, as shown in FIG. 10A, FIG. 10B, and FIG. 10C, it is possible to employ a configuration in which the grasper 4 is opened and closed by fixing one jaw 10b and pivoting the other jaw 10a with two wires 5a and 5b. The two wires 5a and 5b are configured to be alternately pushed and pulled as a pulley 17 is rotated at the proximal end. The ends of the two wires 5a and 5b are fixed to the pulley 17 and the jaws 10b at fixing parts 18 and 19, respectively.

In this case, because the grasping force of the grasper 4 is generated from the difference in tension between the two wires 5a and 5b, the grasping force does not increase simply by equally increasing the tensions in the two wires 5a and 5b by moving the flexible section 2 toward the proximal end, relative to the movable member 3.

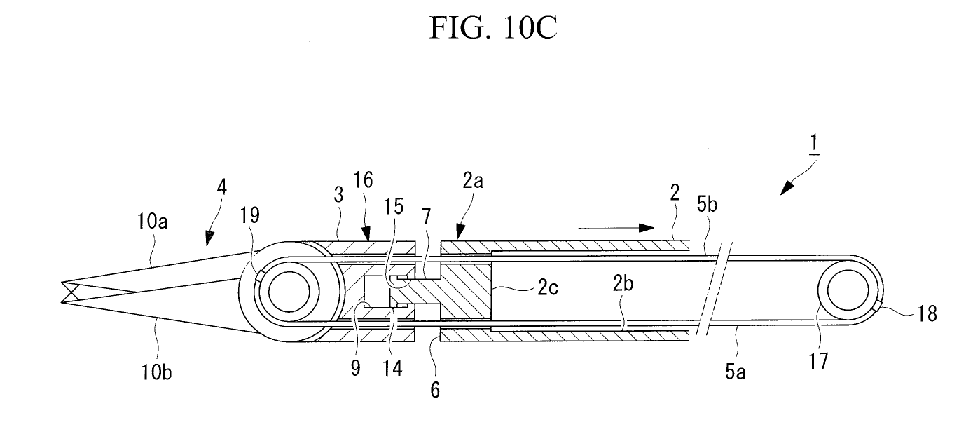

In this structure, as shown in FIG. 10B, when the grasper 4 is closed, the pulley 17 is rotated to pull one wire 5a and to feed out the other wire 5b. Accordingly, as shown by a solid line in FIG. 11, in a state before the grasper 4 is closed, as shown in FIG. 10A, even though initial tensions are applied such that the tensions in the two wires 5a and 5b are equal, when the grasper 4 is closed, as shown by a dashed line in FIG. 11, the tension in one wire 5a decreases, and the tension in the other wire 5b increases.

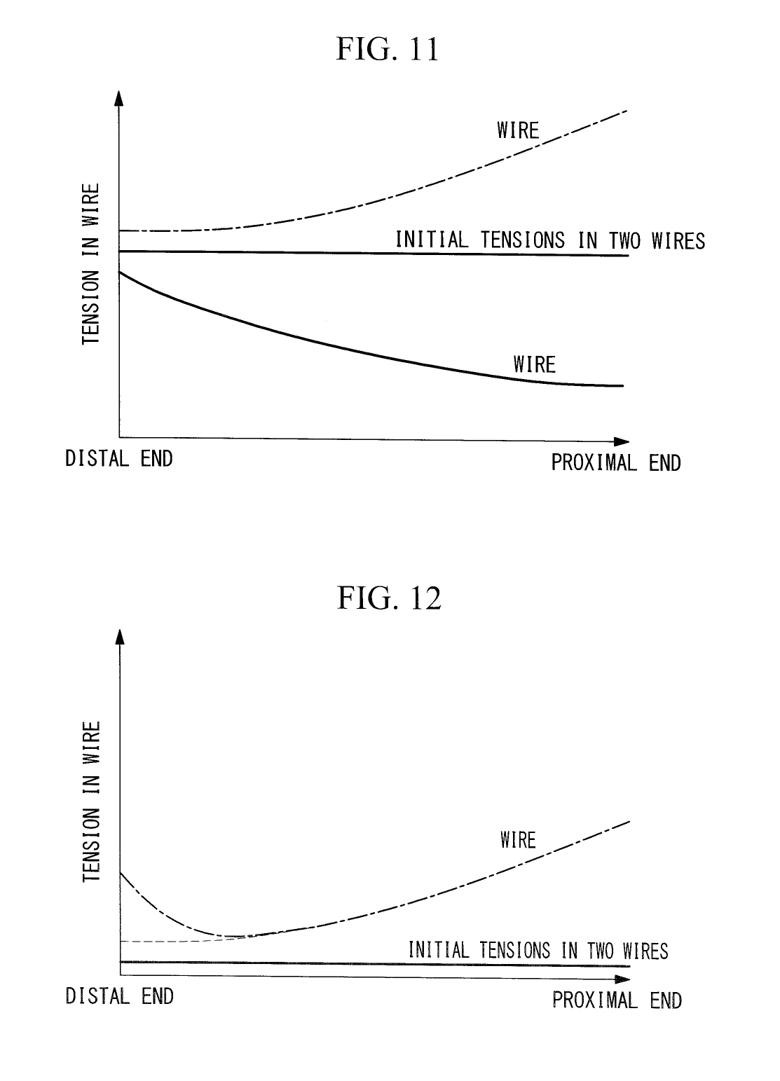

Hence, in this embodiment, as shown by a solid line in FIG. 12, the initial tensions applied to the two wires 5a and 5b are set to be sufficiently low, so that the other wire 5b slackens when one wire 5a is tightened. As a result, as shown in FIG. 10C, when the tension in one wire 5a is increased near the movable member 3 by pulling the flexible section 2 toward the proximal end, relative to the movable member 3, only the slack in the other wire 5b is eliminated, and an increase in tension is suppressed. This leads to an advantage in that it is possible to increase the difference in tension between the two wires 5a and 5b and, thus, to increase the grasping force of the grasper 4.

As a result, the following aspect is read from the above described embodiment of the present invention.

An aspect of the present invention is a medical treatment tool including: a long flexible section; a movable member attached to a distal end of the flexible section so as to be relatively movable in a longitudinal-axis direction of the flexible section; a grasper that is attached to the movable member and that includes a pair of jaws that can be opened and closed, at least one of the jaws being supported so as to be pivotable about a shaft intersecting the longitudinal axis; and a tension transmission that is disposed in the flexible section so as to be able to move forward and backward in the longitudinal-axis direction, that is joined to the jaws at one end, and that transmits tension to pivot the jaws.

According to this aspect, by inserting the grasper and the flexible section into the body from the distal end with the movable member relatively moved toward the long flexible section and by pulling the tension transmission at the proximal end of the flexible section with tissue disposed between the pair of jaws, the pair of jaws are closed, and the tissue inside the body is grasped by the grasper. In this state, it is difficult to maintain a state in which the tissue is grasped by the grasper because, typically, due to the bent flexible section, the tension in the tension transmission is attenuated more at positions closer to the distal end due to the friction between the flexible section and the tension transmission, and thus, sufficient tension is unlikely to act on the grasper.

In this aspect, from this state, by pulling the flexible section toward the proximal end relative to the movable member, the grasper can be relatively moved in the direction away from flexible section, and thus, the tension applied to the tension transmission located at the distal end of the flexible section can be increased without changing the position of the movable member. In other words, according to this aspect, it is possible to easily increase the tension acting on the grasper to maintain a state in which the tissue is grasped by the grasper.

In the above aspect, the movable member may include a first movable part that is attached to the flexible section so as to be relatively movable in the longitudinal-axis direction, and a second movable part that is attached to the first movable part so as to be relatively movable in the longitudinal-axis direction. In a state in which the grasper is maximally brought toward the flexible section, the first movable part, the second movable part, and the flexible section are stacked on top of one another in the longitudinal-axis direction.

With this configuration, a mechanism for increasing the tension in the tension transmission can be formed of a mechanism that can be accommodated in a space shorter than the total of the amount of movement of the first movable part relative to the flexible section and the amount of movement of the second movable part relative to the first movable part. By reducing the length of a rigid portion, smooth insertion of the grasper along a winding path inside the body can be made easy.

Furthermore, in the above aspect, the medical treatment tool may further include an anchoring part that anchors the flexible section and the tension transmission when the flexible section is moved toward the proximal end relative to the movable member with the jaws of the grasper closed.

With this configuration, by anchoring the flexible section and the tension transmission with the anchoring part, the pulling force for moving the flexible section can be easily transmitted to the tension transmission, and thus, the tension in the tension transmission near the grasper can be reliably increased.

Furthermore, in the above aspect, the medical treatment tool may further include a restriction mechanism that restricts the amount of relative movement between the flexible section and the movable member.

With this configuration, even if the flexible section and the movable part are to be relatively moved too much in the directions away from each other, the restriction mechanism restricts the amount of relative movement, and thus, it is possible to prevent excessive tension from being applied to the tension transmission.

Furthermore, in the above aspect, the tension transmission may include a pair of tension transmissions provided in accordance with the pivot directions of the jaws. The tension transmissions are driven such that, when the tension in one tension transmission is increased, the tension in the other tension transmission is reduced. The initial tensions in the tension transmissions may be set such that the difference in tension between the pair of tension transmissions is increased when the flexible section is moved toward the proximal end relative to the movable member.

With this configuration, also in a grasper configured to pivot jaws with a pair of tension transmissions, it is possible to increase the difference in tension between the pair of tension transmissions by means of the relative movement between the movable member and the flexible section and, thus, to increase the grasping force of the grasper.

### REFERENCE SIGNS LIST

1 medical treatment tool
2 flexible section
2a distal end of flexible section
3, 3a, 3b movable member (first and second movable part)
4 grasper
5, 5a, 5b wire (tension transmission)
10a, 10b jaw
13 anchoring part
16 restriction mechanism

1. A medical treatment tool comprising:
a long flexible section;
a movable member attached to a distal end of the flexible section so as to be relatively movable in a longitudinal-axis direction of the flexible section;
a grasper that is attached to the movable member and that includes a pair of jaws that can be opened and closed, at least one of the jaws being supported so as to be pivotable about a shaft intersecting the longitudinal axis; and
a tension transmission that is disposed in the flexible section so as to be able to move forward and backward in the longitudinal-axis direction, that is joined to the jaws at one end, and that transmits tension to pivot the jaws.

2. The medical treatment tool according to claim 1, wherein
the movable member includes a first movable part that is attached to the flexible section so as to be relatively movable in the longitudinal-axis direction, and a second movable part that is attached to the first movable part so as to be relatively movable in the longitudinal-axis direction, and,
in a state in which the grasper is maximally brought toward the flexible section, the first movable part, the second movable part, and the flexible section are stacked on top of one another in the longitudinal-axis direction.

3. The medical treatment tool according to claim 1, further comprising an anchoring part that anchors the flexible section and the tension transmission when the flexible section is moved toward the proximal end relative to the movable member with the jaws of the grasper closed.

4. The medical treatment tool according to claim 1, further comprising a restriction mechanism that restricts the amount of relative movement between the flexible section and the movable member.

5. The medical treatment tool according to claim 1, wherein
the tension transmission includes a pair of tension transmissions provided in accordance with the pivot directions of the jaws, the tension transmissions being driven such that, when the tension in one tension transmission is increased, the tension in the other tension transmission is reduced, and
initial tensions in the tension transmissions being set such that the difference in tension between the pair of tension transmissions is increased when the flexible section is moved toward the proximal end relative to the movable member.
